(19) United States
(12) Patent Application Publication
(10) Pub. No.: US 2012/0010706 A1
(43) Pub. Date: Jan. 12, 2012

(54) NON-ROTATING IMPLANT ASSEMBLY AND BACKING PLATE THEREFOR

(75) Inventor: David J. Schuessler, Ventura, CA (US)

(73) Assignee: ALLERGAN, INC., Irvine, CA (US)

(21) Appl. No.: 13/177,295

(22) Filed: Jul. 6, 2011

Related U.S. Application Data
(60) Provisional application No. 61/361,726, filed on Jul. 6, 2010.

(51) Int. Cl. A61F 2/12

(52) U.S. Cl. 623/8

(57) ABSTRACT

The present specification discloses a backing plate comprising a front surface and a back surface comprising a plurality of parallel ridges and grooves, and soft tissue implants comprising such backing plates.

### RELATED APPLICATION

This application claims the benefit of and priority to U.S. Provisional Patent Application No. 61/361,726, filed on Jul. 6, 2010, the entire specification of which being incorporated herein by this specific reference.

### BACKGROUND

Soft tissue implantable devices or soft tissue implants are commonly used for a wide variety of purposes. One use is as a prosthesis to replace or augment body tissue. The implant serves to support surrounding tissue and to maintain the normal appearance of the body. The restoration of this normal appearance has an extremely beneficial psychological effect on post-operative patients, alleviating much of the shock and depression that often follows extensive surgical procedures.

Soft tissue implants used as prosthesis typically include a flexible and inflatable envelope or shell including an interior chamber and outer surface and a valve or fill port on one side. The interior chamber of the shell is generally filled with a saline, an elastomer, a gel, a foam, combinations of these materials, or other suitable material known in the art to provide a complete fluid-filled prosthesis. Although filling of the shell's interior chamber may take place before or after the implant is positioned in the patient, a prosthesis is typically implanted into a body region in an empty or only partially filled state. The implant is then inflated to its final size by means of the valve. This helps reduce the size of the needed incision, and enables a surgeon to adjust the volume of the implant.

Another use for a soft tissue implant is a tissue expander. Prior to implantation of a soft tissue prosthesis, it is common practice to utilize a tissue expander in order to create the space necessary for the permanent prosthesis. Implantation of a tissue expander stretches the existing skin causing a growth response that stimulates the growth of new skin. While the exact physiologic mechanism of this response remains unclear, clinical success has been reported over many years.

A tissue expander is essentially similar to a prosthesis in that it comprises a flexible and inflatable shell including an interior chamber and outer surface and a valve. Like a prosthesis, the valve may be located on one side of the inflatable shell itself or it may be remotely located and connected to the inflatable shell by means of a conduit. The tissue expander is placed subcutaneously in the patient, at the location where tissue is to be expanded. The valve, whether on the implant or remotely located thereto, is also subcutaneously positioned, and is configured to allow the introduction of fluid, typically saline, into the interior chamber, usually by injection with a syringe. Upon introduction of a predetermined amount of fluid into the chamber of the shell, the skin and subcutaneous tissues overlying the expander stretches in response to the pressure exerted upon such tissues by the inflated shell. After gradual inflation at pre-determined intervals, which may extend over weeks or months, the skin and subcutaneous tissue will expand to the point where further medical procedures can be performed, such as, e.g., permanent implantation of a prosthesis, plastic and reconstructive surgery, or harvesting of the skin and subcutaneous tissue for use at some other body location, like grafting to a skin region damaged by burns, surgery, or congenital deformities.

One problem associated with soft tissue implants is that once surgically placed the implant can rotate, turn, shift, slip or otherwise move from its desired position. This unwanted movement can impede the implant from functioning properly and/or create a cosmetically unacceptable appearance. The present specification addresses this problem by providing a backing plate that reduces or prevents unwanted movement of a soft tissue implant after surgical positioning to its correct anatomical location.

### BRIEF DESCRIPTION OF THE DRAWINGS

FIGS. 1A and 1B are front and back views, respectively, of a backing plate for a mammary prosthesis in accordance with one embodiment disclosed herein. FIG. 1C is an elevation view of the backing plate shown in FIGS. 1A and 1B.

FIG. 2A is an elevation view of a soft tissue implant in accordance with an embodiment disclosed herein. FIG. 2B is an elevation view of a soft tissue implant in accordance with another embodiment of the invention.

FIGS. 3A-3J are elevation views of further embodiment disclosed herein.

FIGS. 4A-4J are still further embodiments disclosed herein.

FIGS. 5A-5C are front, back, and elevation views, respectively, of a backing plate including a valve, in accordance with another embodiment disclosed herein. FIG. 5D is an elevation view of a soft tissue implant including the backing plate shown in FIGS. 5A-5C.

FIGS. 6A-6C are front, back, and elevation views, respectively, of a backing plate including a valve, in accordance with yet another embodiment disclosed herein. FIG. 6D is an elevation view of a soft tissue implant including the backing plate shown in FIGS. 6A-6C.

FIGS. 7A-7B are cross-sectional views of a valve portion of the backing plate shown in FIGS. 6A-6C.

FIGS. 8A-8C illustrate various views of another backing plate disclosed herein.

FIG. 8D is an elevation view of the backing plate of FIG. 8A-8C as part of a breast implant assembly.

FIGS. 9A-9C illustrate various views of yet another backing plate disclosed herein.

FIG. 9D is an elevation view of the backing plate of FIG. 9A-9C as part of a breast implant assembly.

FIGS. 10A-10C show various views of a further backing plate disclosed herein.

FIGS. 11A-11C illustrate yet another backing plate disclosed herein.

FIG. 11D is a elevation view of the backing plate shown in FIGS. 11A-11C as part of a breast implant assembly.

FIG. 12 is a perspective view of an even further backing plate disclosed herein.

### DETAILED DESCRIPTION

The present specification discloses a prosthesis or implant, for example a breast prosthesis or breast implant, comprising an elastomeric anterior sposterior portion or shell and a posterior portion, secured to the anterior portion and comprising a backing plate that reduces or prevents unwanted movement of a soft tissue implant after surgical positioning.

The backing plate comprises a front surface and a back surface comprising a surface defined by spaced apart ridges, for example, substantially parallel ridges. The spacing between the ridges may be referred to sometimes herein as “grooves”.

In some embodiments, the structure of the backing plate allows the implant to resist unwanted movement because the ridges and grooves engage and hold the implant against the underlying tissue, possibly due to the mechanical pressure exerted on the implant by the overlying tissue. The engaged ridges and grooves enable the implant to resist mechanical forces generated from, e.g., body movements or body impacts, that would otherwise cause the implant to rotate, turn, shift, slip, or otherwise move from its desired position. Advantageously, the backing plate may be sufficiently flexible, for example, in a direction aligned with the alignment of the ridges, so as to facilitate rolling or folding of the implant, for example, in the direction of the ridges, so as to facilitate orientation and insertion of the implant during surgery, as well as its subsequent removal.

The spacing between the ridges and grooves may be of equal length or varied length, and if varied, the spacing pattern may be defined by a mathematical function or the spacing pattern may be irregular. In addition, the ridge height, ridge width, and/or groove width can be of equal length or varied length, and if varied, the spacing pattern may be defined by a mathematical function or the spacing pattern may be irregular. Further, the contour of the ridges may be a curved shape, a polygonal shape, or any combination thereof. The backing plate may be composed of material that is substantially non-degradable and biocompatible including, without limitation, a medical-grade silicon-based elastomer.

Other aspects of the present specification disclose a soft tissue implant comprising a backing plate disclosed in the present specification and an implant shell having an interior chamber. The soft tissue implant can be, without limitation, a relatively more permanent prosthetic implant or a relatively temporary tissue expander. The implant shell may be composed of material that is substantially non-degradable and biocompatible including, without limitation, a medical-grade silicon-based elastomer.

In one embodiment of the invention, a breast prosthesis is provided, the prosthesis comprising a fillable, elastic anterior portion, and a flexible backing plate, secured to the anterior portion and forming a posterior portion of the prosthesis, the anterior portion and the backing plate defining a fluid-fillable cavity therebetween. The backing plate comprises a surface defined by a plurality of alternating ridges, the ridges having at least one of varied heights or varied widths and having a flexibility between the ridges to enable the backing plate to form a substantially rolled configuration. In addition, the backing further comprising a valve for facilitating filling of the chamber.

The prosthesis may be comprised of any substantially non-degradable and biocompatible material suitable for the intended purpose. As used herein, the term “non-degradable” refers to a material that is not prone to degrading, decomposing, or breaking down to any substantial or significant degree while implanted in a host. Non-limiting examples of substantial non-degradation include less than 10% degradation of a backing plate over a time period measured, less than 5% degradation of a backing plate over a time period measured, less than 3% degradation of a backing plate over a time period measured, less than 1% degradation of a backing plate over a time period measured. As used herein, the term “biocompatible” refers to a material's ability to perform its intended function, with a desired degree of incorporation in the host, without eliciting any undesirable local or systemic effects in that host.

Suitable substantially non-degradable and biocompatible materials include, without limitation, elastomers. As used herein, the term “elastomer” or “elastic polymer” refers to an amorphous polymer that exists above its glass transition temperature (Tg) at ambient temperatures, thereby conferring the property of viscoelasticity so that considerable segmental motion is possible, and includes, without limitation, carbon-based elastomers, silicon-based elastomers, thermoset elastomers, and thermoplastic elastomers. As used herein, the term “ambient temperature” refers to a temperature of about 18° C. to about 22° C. Elastomers, ether naturally-occurring or synthetically-made, comprise monomers commonly made of carbon, hydrogen, oxygen, and/or silicon which are linked together to form long polymer chains. Elastomers are typically covalently cross-linked to one another, although non-covalently cross-linked elastomers are known. Elastomers may be homopolymers or copolymers, degradable, substantially non-degradable, or non-degradable. Copolymers may be random copolymers, blocked copolymers, graft copolymers, and/or mixtures thereof. Unlike other polymers classes, an elastomer can be stretched many times its original length without breaking by reconfiguring themselves to distribute an applied stress, and the cross-linkages ensure that the elastomers will return to their original configuration when the stress is removed. Elastomers can be a non-medical grade elastomer or a medical grade elastomer. Medical grade elastomers are typically divided into three categories: non implantable, short term implantable and long-term implantable. Exemplary substantially non-degradable and/or non-degradable, biocompatible, elastomers include, without limitation, bromo isobutylene isoprene (BIIR), polybutadiene (BR), chloro isobutylene isoprene (CIIR), polychloroprene (CR), chlorosulphonated polyethylene (CSM), ethylene propylene (EP), ethylene propylene diene monomer (EPDM), fluoronated hydrocarbon (FKM), fluoro silicone (FVQM), hydrogenated nitrile butadiene (HNBR), polyisoprene (IR), isobutylene isoprene butyl (IIR), methyl vinyl silicone (MVQ), acrylonitrile butadiene (NBR), polyurethane (PU), styrene butadiene (SBR), styrene ethylene/butylene styrene (SEBS), polydimethylsiloxane (PDMS), polysiloxane (SI), and acrylonitrile butadiene carboxy monomer (XNBR).

One particular elastomer useful as a material for the backing plate is a silicon-based elastomer. As used herein, the term “silicon-based elastomer” refers to any silicon containing elastomer, such as, e.g., methyl vinyl silicone, polydimethylsiloxane, or polysiloxane. A silicone-based elastomer can be a high temperature vulcanization (HTV) silicone or a room temperature vulcanization (RTV). A silicon-based elastomer can be a non-medical grade silicon-based elastomer or a medical grade silicon-based elastomer. As used herein, the term “medical grade silicon-based elastomer” refers to a silicon-based elastomer approved by the U.S. Pharmacopedia (USP) as at least Class V. Medical grade silicon-based elastomers are typically divided into three categories: non implantable, short term implantable and long-term implantable.

The substantially non-degradable and biocompatible materials used to make a backing plate disclosed in the present specification may, or may not, be a material that also provides sufficient structural support to direct the pressure exerted by the fluid outward towards the overlaying tissue. This outward direction 1) maximizes the pressure exerted on the overlaying tissue, thereby facilitating the expansion of the tissue at the desired location and 2) decreases the pressure exerted on the underlying tissue, thereby reducing discomfort and pain. A backing plate providing sufficient structural support is useful when the plate is incorporated in a tissue expander.

The substantially non-degradable and biocompatible materials used to make a backing plate disclosed in the present specification may, or may not, be a material that also provides sufficient resistance to prevent puncture through the backing plate by a sharp implement such as, e.g., a syringe needle or scalpel. A puncture-resistant backing plate is useful when incorporated into a soft tissue implant where the implant comprises a self-sealing shell. See, e.g., Schuessler, Self-Sealing Shell for Inflatable Prostheses, U.S. 2010/0049317; Schuessler, Self-Sealing Shell for Inflatable Prostheses, U.S. 2010/0049316; each of which is incorporated by reference in its entirety for the purpose of describing and disclosing self-sealing implant shells.

In an embodiment, a backing plate is substantially non-degradable. In aspects of this embodiment, a backing plate is substantially non-degradable for, e.g., about five years, about ten years, about 15 years, about 20 years, about 25 years, about 30 years, about 35 years, about 40 years, about 45 years, or about 50 years. In other aspects of this embodiment, a backing plate is substantially non-degradable for, e.g., at least five years, at least ten years, at least 15 years, at least 20 years, at least 25 years, at least 30 years, at least 35 years, at least 40 years, at least 45 years, or at least 50 years. In yet other aspects of this embodiment, a backing plate exhibits less than 5% degradation, less than 3% degradation, or less than 1% degradation over for, e.g., about five years, about ten years, about 15 years, about 20 years, about 25 years, about 30 years, about 35 years, about 40 years, about 45 years, or about 50 years. In still other aspects of this embodiment, a backing plate exhibits less than 5% degradation, less than 3% degradation, or less than 1% degradation over for, e.g., at least five years, at least ten years, at least 15 years, at least 20 years, at least 25 years, at least 30 years, at least 35 years, at least 40 years, at least 45 years, or at least 50 years.

In another embodiment, a backing plate is substantially biocompatible. In aspects of this embodiment, a backing plate is biocompatible for, e.g., at least five years, at least ten years, at least 15 years, at least 20 years, at least 25 years, at least 30 years, at least 35 years, at least 40 years, at least 45 years, or at least 50 years.

In yet another embodiment, a backing plate is composed of a medical grade elastomer. In aspects of this embodiment, a medical grade elastomer is, e.g., a medical grade carbon-based elastomer, a medical grade silicon-based elastomer, a medical grade thermoset elastomer, or a medical grade thermoplastic elastomer. In other aspects of this embodiment, an elastomer is, e.g., a medical grade, long-term implantable, carbon-based elastomer, a medical grade, long-term implantable, silicon-based elastomer, a medical grade, long-term implantable, thermoset elastomer, or a medical grade, long-term implantable, thermoplastic elastomer. In still other aspects, a medical grade elastomer is, e.g., a medical grade bromo isobutylene isoprene, a medical grade polybutadiene, a medical grade chloro isobutylene isoprene, a medical grade polychloroprene, a medical grade chlorosulphonated polyethylene, a medical grade ethylene propylene, a medical grade ethylene propylene diene monomer, a medical grade fluoronated hydrocarbon, a medical grade fluoro silicone, a medical grade hydrogenated nitrile butadiene, a medical grade polyisoprene, a medical grade isobutylene isoprene butyl, a medical grade methyl vinyl silicone, a medical grade acrylonitrile butadiene, a medical grade polyurethane, a medical grade styrene butadiene, a medical grade styrene ethylene/butylene styrene, a medical grade polydimethylsiloxane, a medical grade polysiloxane, or a medical grade acrylonitrile butadiene carboxy monomer.

In another embodiment, a backing plate is composed of a silicon-based elastomer. In an aspect of this embodiment, a silicon-based elastomer is a medical grade silicon-based elastomer. In aspects of this embodiment, a medical grade silicon-based elastomer is, e.g., at least a USP Class V silicon-based elastomer, at least a USP Class VI silicon-based elastomer, or USP Class VII silicon-based elastomer. In yet other aspects, a medical grade silicon-based elastomer is a long-term implantable silicon-based elastomer. In yet other aspects, a medical grade silicon-based elastomer is, e.g., a medical grade, long-term implantable, methyl vinyl silicone, a medical grade, long-term implantable, polydimethylsiloxane, or a medical grade, long-term implantable, polysiloxane.

A backing plate disclosed in the present specification may be of any outline or perimeter, with the proviso that the plate outline or perimeter is sufficient to facilitate engagement of the underlying tissue and resist the mechanical forces that would cause the implant to rotate, turn, shift, slip, or otherwise move from its desired position. In one embodiment, the plate outline or perimeter conforms to the general shape of an implant surface that come in contact with underlying tissue, such as, e.g., surface areas of underlying tissue for implants of a breast, pectoral area, upper arm (triceps and biceps), buttock, upper leg (quadriceps and hamstring), and calf. In another embodiment, a plate outline or perimeter can be a geometric outline, such as, e.g., a closed curved outline like a circle or ellipse, or a polygonal outline like a triangle, a quadrilateral, pentagon, hexagon, or other polygonal shape.

A backing plate disclosed in the present specification may be of any planar shape, with the proviso that the planar shape is sufficient to facilitate engagement of the underlying tissue and resist the mechanical forces that would cause the implant to rotate, turn, shift, slip, or otherwise move from its desired position. Thus, a backing plate may be flat plate, a plate having an open curved shape in a uniform manner, or planar shape that conforms to the curvature of the underlying tissue for which the implant will be positioned. Thus, in one embodiment, a backing plate is flat. In another embodiment, a backing plate is in an open curved shape. In aspects of this embodiment, a backing plate is uniformly convex in shape, irregularly convex in shape, uniformly concave in shape, or irregularly concave in shape. In another embodiment, a backing plate is in shaped to conform to the curvature of the underlying tissue for which the implant will be positioned.

A backing plate disclosed in the present specification may be of any thickness, with the proviso that the planar shape is sufficient to facilitate engagement of the underlying tissue and resist the mechanical forces that would cause the implant to rotate, turn, shift, slip, or otherwise move from its desired position. Thus, a backing plate may be of substantially uniform thickness or may be varied in thickness. In one aspect of this embodiment, a backing plate has a thickness that is greatest in the middle and uniformly tappers out to a thinner thickness at the periphery of the backing plate. In other aspects of this embodiment, a backing plate can have a thickness of, e.g., at least 1 mm in thickness, at least 2 mm in thickness, at least 3 mm in thickness, at least 4 mm in thickness, at least 5 mm in thickness, at least 6 mm in thickness, at least 7 mm in thickness, at least 8 mm in thickness, at least 9 mm in thickness, or at least 10 mm in thickness. In other aspects of this embodiment, a backing plate can have a thickness of, e.g., about 1 mm to about 15 mm, about 2 mm to about 15 mm, about 3 mm to about 15 mm, about 4 mm to about 15 mm, about 5 mm to about 15 mm, about 6 mm to about 15 mm, about 7 mm to about 15 mm, about 8 mm to about 15 mm, about 1 mm to about 10 mm, about 2 mm to about 10 mm, about 3 mm to about 10 mm, about 4 mm to about 10 mm, about 5 mm to about 10 mm, about 6 mm to about 10 mm, about 7 mm to about 10 mm, about 1 mm to about 8 mm, about 2 mm to about 8 mm, about 3 mm to about 8 mm, about 4 mm to about 8 mm, about 5 mm to about 8 mm, about 1 mm to about 6 mm, about 2 mm to about 6 mm, about 3 mm to about 6 mm, or about 4 mm to about 6 mm, where the backing plate if thickest in the center and thinnest at the periphery.

A backing plate disclosed in the present specification may be made as a single piece or may comprise a plurality of pieces, with the proviso that the plate or plates are sufficient to facilitate engagement of the underlying tissue and resist the mechanical forces that would cause the implant to rotate, turn, shift, slip, or otherwise move from its desired position. The plurality of backing plates may integrate with one another forming a backing plate having an outline or perimeter that conforms to the general shape of an implant surface that come in contact with underlying tissue or a geometric shape as described above. Alternatively, plurality of backing plates may each be of different outlines, shapes, and sizes that are independent of one another. Integration of backing plates can be achieved simply by placing the plates next to one another by affixing the plates to an implant shell, or by affixing the plates to one another, such as, e.g., with hinges. A soft tissue implant comprising ridges which may be one piece or may be a plurality of separate pieces allows the implant to be flexible at the junctures or junctures between ridges or pieces while still providing the required structural support necessary to direct the pressure exerted by the fluid outward towards the overlaying tissue. In aspects of this embodiment, a backing plate comprises, e.g., two pieces, three pieces, four pieces, or five pieces.

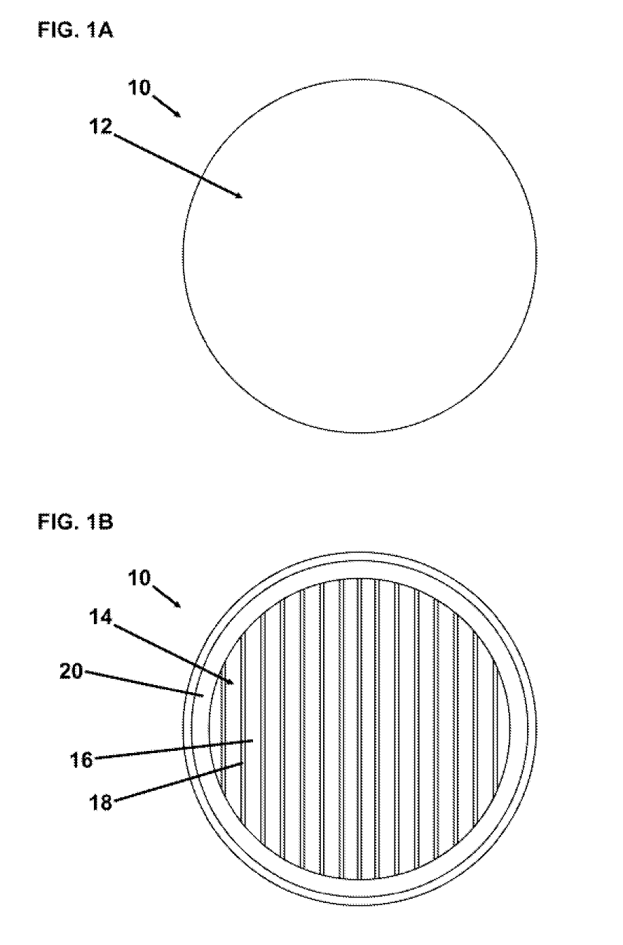

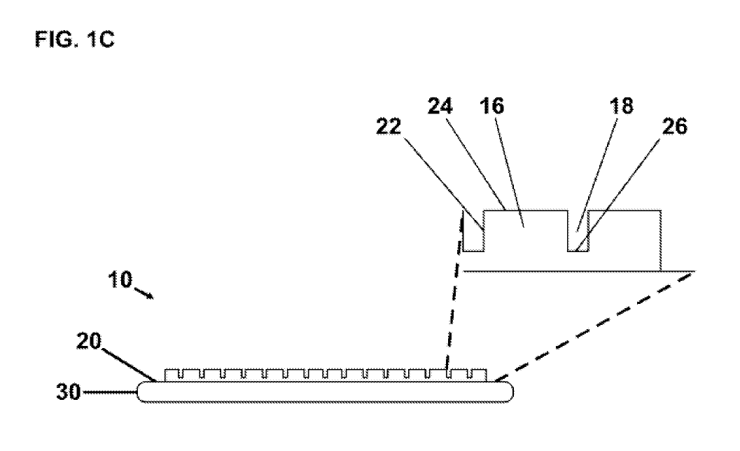

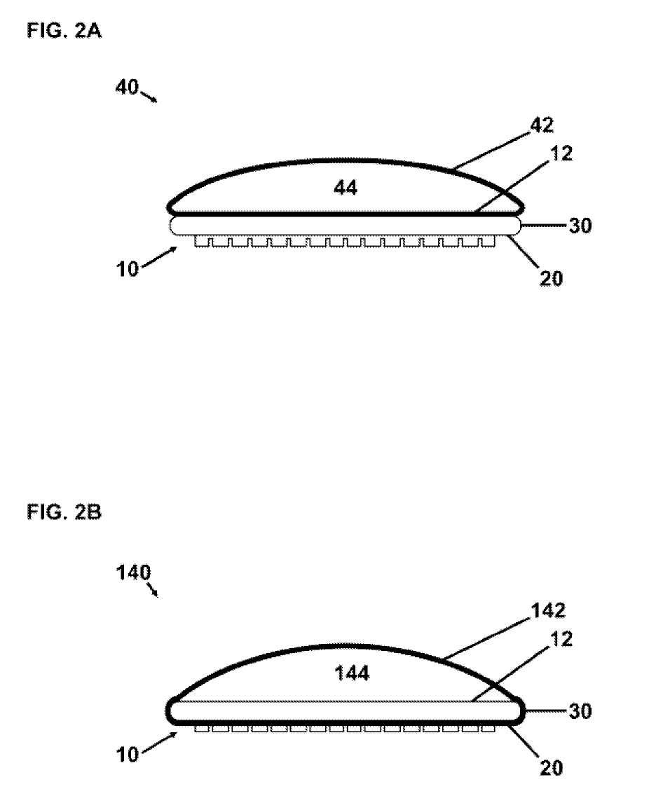

Aspects of the present specification disclose, in part, backing plate having a front surface. FIG. 1A is a front view showing the front surface 12 of the backing plate 10. The front surface provides a contact area where an implant can be affixed to the backing plate. In some embodiments, an implant shell is affixed to all or a portion of front surface 12. The front surface area may be smooth or may be textured to facilitate attachment of the implant. As discussed below, the front surface may have a conduit opening. In one embodiment, the front surface serves as an attachment surface used to affix a shell of the implant (FIG. 2A). FIG. 2A is a elevation view showing a soft tissue implant 40 comprising a shell 42 having an interior chamber 44 that is affixed to front surface 12 of backing plate 10. In another embodiment, a shell of the implant is affixed to the front surface in a manner that incorporates at least a portion of the front surface as an integral part of the interior chamber of the shell (FIG. 2B). FIG. 2B is a elevation view showing a soft tissue implant 140 comprising a shell 142 having an interior chamber 144 that is affixed to side 30 of backing plate 10 and/or outside rim 20 of back surface 14.

Aspects of the present specification disclose, in part, backing plate having a back surface pattern comprising a plurality of parallel ridges and grooves. FIG. 1B is a back view showing the back surface 14 of backing plate 10 including the parallel ridges 16 and grooves 18 and the outside rim 20. As used herein, the term “ridge” refers to the raised or elevated portion of the back surface of a backing plate having a height aspect and a width aspect. As used herein, the term “groove” refers to the depression or channel portion of the back surface of a backing plate formed by the width of adjacent ridges. FIG. 1C is an elevation view of the backing plate 10 and shows the height 22 and width 24 of ridges 16 and the width 26 of grooves 18. This view also shows side 30 of backing plate 10. In some embodiments, an implant shell is affixed to side 30 of backing plate 10 and/or outside rim 20 of back surface 14. A back surface pattern can have any number of parallel ridges and grooves, with the proviso that the number of parallel ridges and grooves present is sufficient to facilitate engagement of the underlying tissue and resist the mechanical forces that would cause the implant to rotate, turn, shift, slip, or otherwise move from its desired position.

In aspects of this embodiment, a backing plate has a back surface pattern comprising, e.g., 2 or more parallel ridges and grooves, 3 or more parallel ridges and grooves, 4 or more parallel ridges and grooves, 5 or more parallel ridges and grooves, 6 or more parallel ridges and grooves, 7 or more parallel ridges and grooves, 8 or more parallel ridges and grooves, 9 or more parallel ridges and grooves, 10 or more parallel ridges and grooves, 11 or more parallel ridges and grooves, 12 or more parallel ridges and grooves, 13 or more parallel ridges and grooves, 14 or more parallel ridges and grooves, 15 or more parallel ridges and grooves, 10 or more parallel ridges and grooves, 10 or more parallel ridges and grooves, 16 or more parallel ridges and grooves, 17 or more parallel ridges and grooves, 18 or more parallel ridges and grooves, 19 or more parallel ridges and grooves, 20 or more parallel ridges and grooves, 25 or more parallel ridges and grooves, 30 or more parallel ridges and grooves, 35 or more parallel ridges and grooves, 40 or more parallel ridges and grooves, 45 or more parallel ridges and grooves, or 50 or more parallel ridges and grooves.

In other aspects of this embodiment, a backing plate has a back surface pattern comprising, e.g., no more than 2 parallel ridges and grooves, no more than 3 parallel ridges and grooves, no more than 4 parallel ridges and grooves, no more than 5 parallel ridges and grooves, no more than 6 parallel ridges and grooves, no more than 7 parallel ridges and grooves, no more than 8 parallel ridges and grooves, no more than 9 parallel ridges and grooves, no more than 10 parallel ridges and grooves, no more than 11 parallel ridges and grooves, no more than 12 parallel ridges and grooves, no more than 13 parallel ridges and grooves, no more than 14 parallel ridges and grooves, no more than 15 parallel ridges and grooves, no more than 16 parallel ridges and grooves, no more than 17 parallel ridges and grooves, no more than 18 parallel ridges and grooves, no more than 19 parallel ridges and grooves, no more than 20 parallel ridges and grooves, no more than 25 parallel ridges and grooves, no more than 30 parallel ridges and grooves, no more than 35 parallel ridges and grooves, no more than 40 parallel ridges and grooves, no more than 45 parallel ridges and grooves, or no more than 50 parallel ridges and grooves.

In yet other aspects of this embodiment, a backing plate has a back surface pattern comprising, e.g., about 2 to about 20 parallel ridges and grooves, about 3 to about 20 parallel ridges and grooves, about 4 to about 20 parallel ridges and grooves, about 5 to about 20 parallel ridges and grooves, about 6 to about 20 parallel ridges and grooves, about 7 to about 20 parallel ridges and grooves, about 8 to about 20 parallel ridges and grooves, about 9 to about 20 parallel ridges and grooves, about 10 to about 20 parallel ridges and grooves, about 12 to about 20 parallel ridges and grooves, about 15 to about 20 parallel ridges and grooves, about 10 to about 50 parallel ridges and grooves, about 15 to about 50 parallel ridges and grooves, about 20 to about 50 parallel ridges and grooves, about 25 to about 50 parallel ridges and grooves, about 30 to about 50 parallel ridges and grooves, about 35 to about 50 parallel ridges and grooves, about 40 to about 50 parallel ridges and grooves, or about 45 to about 50 parallel ridges and grooves.

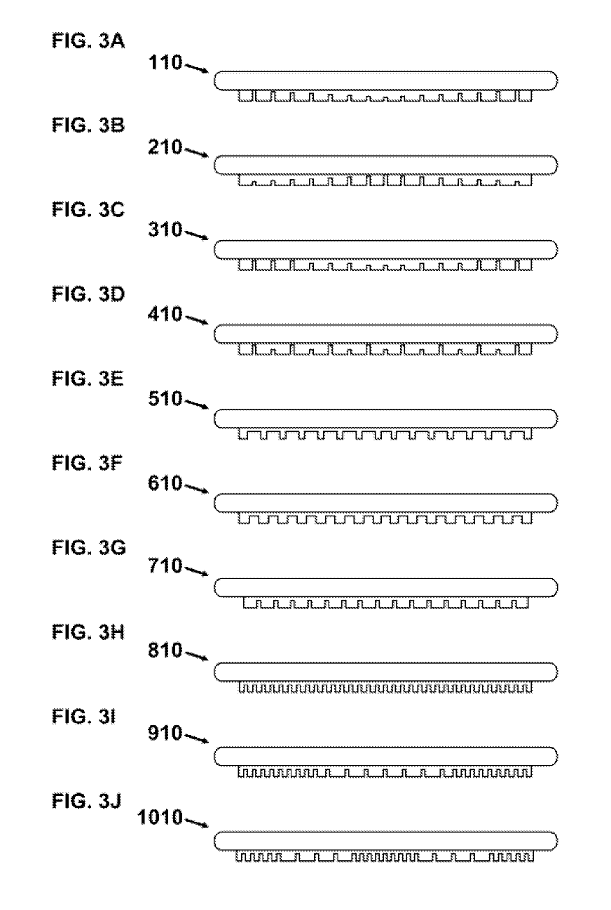

A ridge can be of any height or width, with the proviso that the height or width of the ridge is sufficient to facilitate engagement of the underlying tissue and resist the mechanical forces that would cause the implant to rotate, turn, shift, slip, or otherwise move from its desired position. The height of each ridge can be of equal length to one another or the height of each ridge can vary in length. If the ridge height is varied, the pattern of height variation can be a pattern defined by a mathematical function such as, e.g., an algebraic function like a polynomial function, a nth root function, or a rational function; a transcendental function like an exponential function, a hyperbolic function, or a logarithmic function; a power function; or a periodic function; or the pattern can be irregular. Similarly, the width of each ridge can be of an equal length to one another or the width of each ridge can vary in length. If the ridge width is varied, the pattern of ridge width variation can be a pattern defined by a mathematical function, or the pattern can be irregular. FIG. 3 shows exemplary back surface patterns having ridges of various heights and/or widths for a backing plate disclosed in the present specification. FIGS. 3A-3D show backing plates 110, 210, 310, and 410 where back surface has varied heights of ridges. FIGS. 3E-3H show backing plates 510, 610, 710, and 810 where back surfaces have varied lengths in ridge widths and/or lengths in groove widths.

In aspects of this embodiment, a ridge has a height of, e.g., about 0.5 mm, about 1 mm, about 1.5 mm, about 2 mm, about 2.5 mm, about 3 mm, about 3.5 mm, about 4 mm, about 4.5 mm, or about 5 mm. In other aspects of this embodiment, a ridge has a height of, e.g., at least 0.5 mm, at least 1 mm, at least 1.5 mm, at least 2 mm, at least 2.5 mm, at least 3 mm, at least 3.5 mm, at least 4 mm, at least 4.5 mm, or at least 5 mm. In yet other aspects of this embodiment, a ridge has a height of, e.g., at most 0.5 mm, at most 1 mm, at most 1.5 mm, at most 2 mm, at most 2.5 mm, at most 3 mm, at most 3.5 mm, at most 4 mm, at most 4.5 mm, or at most 5 mm. In still other aspects of this embodiment, a ridge has a height of, e.g., about 0.5 mm to about 5 mm, about 1 mm to about 5 mm, about 1.5 mm to about 5 mm, about 0.5 mm to about 4 mm, about 0.5 mm to about 3 mm, about 0.5 mm to about 2 mm, or about 0.5 mm to about 1.5 mm.

In further aspects of this embodiment, a ridge has a width of, e.g., about 0.5 mm, about 1 mm, about 1.5 mm, about 2 mm, about 2.5 mm, about 3 mm, about 3.5 mm, about 4 mm, about 4.5 mm, or about 5 mm. In other aspects of this embodiment, a ridge has a width of, e.g., at least 0.5 mm, at least 1 mm, at least 1.5 mm, at least 2 mm, at least 2.5 mm, at least 3 mm, at least 3.5 mm, at least 4 mm, at least 4.5 mm, or at least 5 mm. In yet other aspects of this embodiment, a ridge has a width of, e.g., at most 0.5 mm, at most 1 mm, at most 1.5 mm, at most 2 mm, at most 2.5 mm, at most 3 mm, at most 3.5 mm, at most 4 mm, at most 4.5 mm, or at most 5 mm. In still other aspects of this embodiment, a ridge has a width of, e.g., about 0.5 mm to about 5 mm, about 1 mm to about 5 mm, about 1.5 mm to about 5 mm, about 0.5 mm to about 4 mm, about 0.5 mm to about 3 mm, about 0.5 mm to about 2 mm, or about 0.5 mm to about 1.5 mm.

In another aspect of this embodiment, the height of each ridge present on the back surface of the backing plate is of an equal length. In yet another of this embodiment, the height of each ridge present on the back surface of the backing plate is of a varied length, the height pattern defined by a mathematical function. In still another aspect of this embodiment, the height of each ridge present on the back surface of the backing plate is of a varied length, the height pattern being irregular.

In another aspect of this embodiment, the width of each ridge present on the back surface of the backing plate is of an equal length. In yet another of this embodiment, the width of each ridge present on the back surface of the backing plate is of a varied length, the ridge width pattern defined by a mathematical function. In still another aspect of this embodiment, the width of each ridge present on the back surface of the backing plate is of a varied length, the ridge width pattern being irregular.

A groove can be of any width, with the proviso that the width of the groove is sufficient to facilitate engagement of the underlying tissue and resist the mechanical forces that would cause the implant to rotate, turn, shift, slip, or otherwise move from its desired position. The width of each groove can be of equal length to one another or the width of each groove can vary in length. If the groove width is varied, the pattern of groove width variation can be a pattern defined by a mathematical function such as, e.g., an algebraic function like a polynomial function, a nth root function, or a rational function; a transcendental function like an exponential function, a hyperbolic function, or a logarithmic function; a power function; or a periodic function; or the pattern can be irregular. FIG. 3 shows exemplary back surface patterns having grooves of various widths for a backing plate disclosed in the present specification. FIGS. 3E-3H show backing plates 510, 610, 710, and 810 where back surfaces have varied lengths in ridge widths and/or lengths in groove widths.

The spacing between individual ridges is determined by the groove width and can be of any length with the proviso that the length is sufficient to facilitate engagement of the underlying tissue and resist the mechanical forces that would cause the implant to rotate, turn, shift, slip, or otherwise move from its desired position. The spacing between ridges and grooves can be of equal length to one another or the spacing between ridges and grooves can vary in length. If the spacing is varied, the pattern of space variation can be a pattern defined by a mathematical function such as, e.g., an algebraic function like a polynomial function, a nth root function, or a rational function; a transcendental function like an exponential function, a hyperbolic function, or a logarithmic function; a power function; or a periodic function; or the pattern can be irregular.

In aspects of this embodiment, a groove has a width of, e.g., about 0.5 mm, about 1 mm, about 1.5 mm, about 2 mm, about 2.5 mm, about 3 mm, about 3.5 mm, about 4 mm, about 4.5 mm, or about 5 mm. In other aspects of this embodiment, a groove has a width of, e.g., at least 0.5 mm, at least 1 mm, at least 1.5 mm, at least 2 mm, at least 2.5 mm, at least 3 mm, at least 3.5 mm, at least 4 mm, at least 4.5 mm, or at least 5 mm. In yet other aspects of this embodiment, a groove has a width of, e.g., at most 0.5 mm, at most 1 mm, at most 1.5 mm, at most 2 mm, at most 2.5 mm, at most 3 mm, at most 3.5 mm, at most 4 mm, at most 4.5 mm, or at most 5 mm. In still other aspects of this embodiment, a groove has a width of, e.g., about 0.5 mm to about 5 mm, about 1 mm to about 5 mm, about 1.5 mm to about 5 mm, about 0.5 mm to about 4 mm, about 0.5 mm to about 3 mm, about 0.5 mm to about 2 mm, or about 0.5 mm to about 1.5 mm.

In another aspect of this embodiment, the width of each groove present on the back surface of the backing plate is of an equal length. In yet another of this embodiment, the width of each groove present on the back surface of the backing plate is of a varied length, the groove width pattern defined by a mathematical function. In still another aspect of this embodiment, the width of each groove present on the back surface of the backing plate is of a varied length, the groove width pattern being irregular.

In yet another aspect of this embodiment, the spacing between ridges and grooves present on the back surface of the backing plate is of an equal length. In yet another of this embodiment, the spacing between ridges and grooves present on the back surface of the backing plate is of a varied length, the spacing pattern defined by a mathematical function. In still another aspect of this embodiment, the spacing between ridges and grooves present on the back surface of the backing plate is of a varied length, the spacing pattern being irregular. FIG. 3 shows exemplary spacing patterns of ridges and grooves of a back surface from a backing plate disclosed in the present specification. FIGS. 3I-3J show backing plates 901 and 1010 where back surfaces have multiple types of ridges and grooves where each type has different lengths in ridge widths and/or lengths in groove widths.

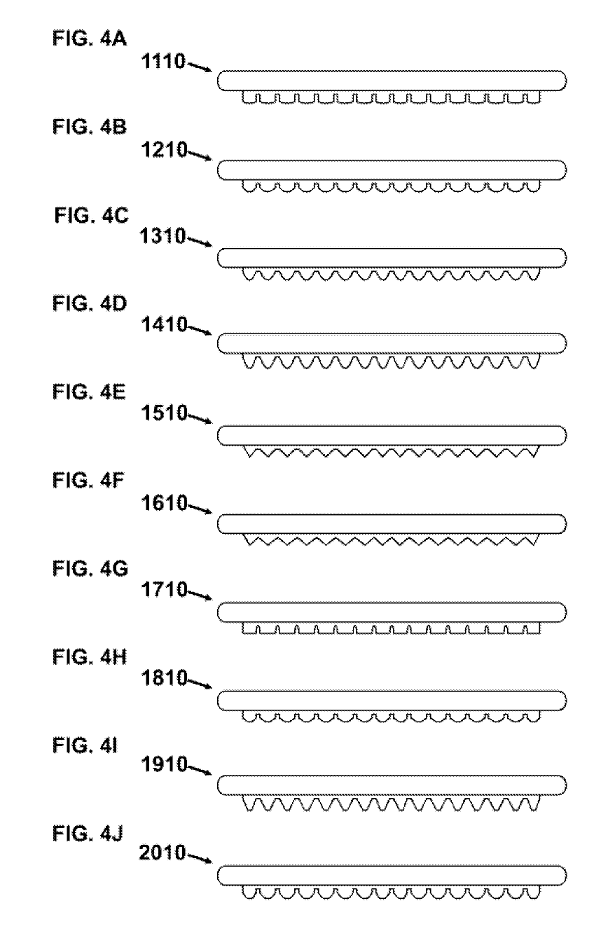

The contour of a ridge or groove can be of any shape with the proviso that the contour is sufficient to facilitate engagement of the underlying tissue and resist the mechanical forces that would cause the implant to rotate, turn, shift, slip, or otherwise move from its desired position. In aspects of this embodiment, the contour of a ridge can be, e.g., a curved shape like a circular shape or elliptical shape, a polygonal shape like a triangular shape, a quadrangular shape, a pentagonal shape, a hexagonal shape, or other polygonal shapes, or any combination thereof. In other aspects of this embodiment, the contour of a groove can be, e.g., a curved shape like a circular shape or elliptical shape, a polygonal shape like a triangular shape, a quadrangular shape, a pentagonal shape, a hexagonal shape, or other polygonal shapes, or any combination thereof. FIG. 4 shows exemplary contours of ridges and grooves of a back surface from a backing plate disclosed in the present specification. FIGS. 4A-4D show backing plates 1110, 1210, 1310, and 1410 where back surfaces have curve shaped ridges. FIGS. 4E-4J show backing plates 1510, 1610, 1710, 1810, 1910, and 2010 where back surfaces have polygonal shaped ridges.

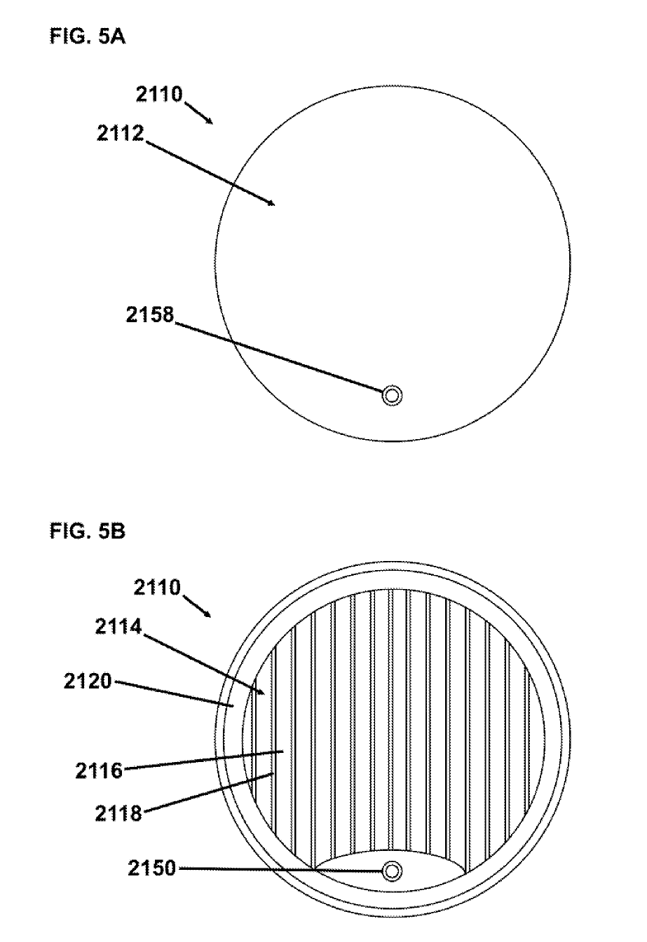

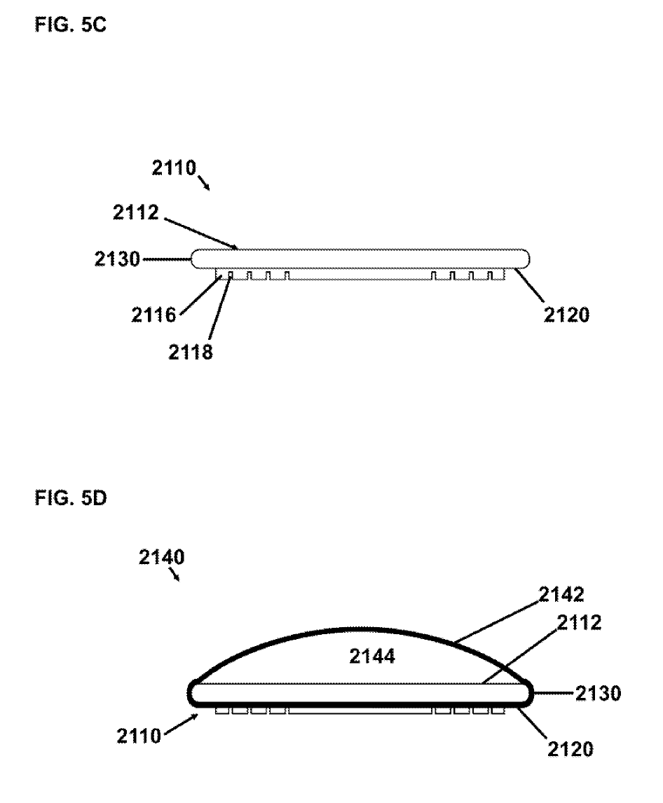

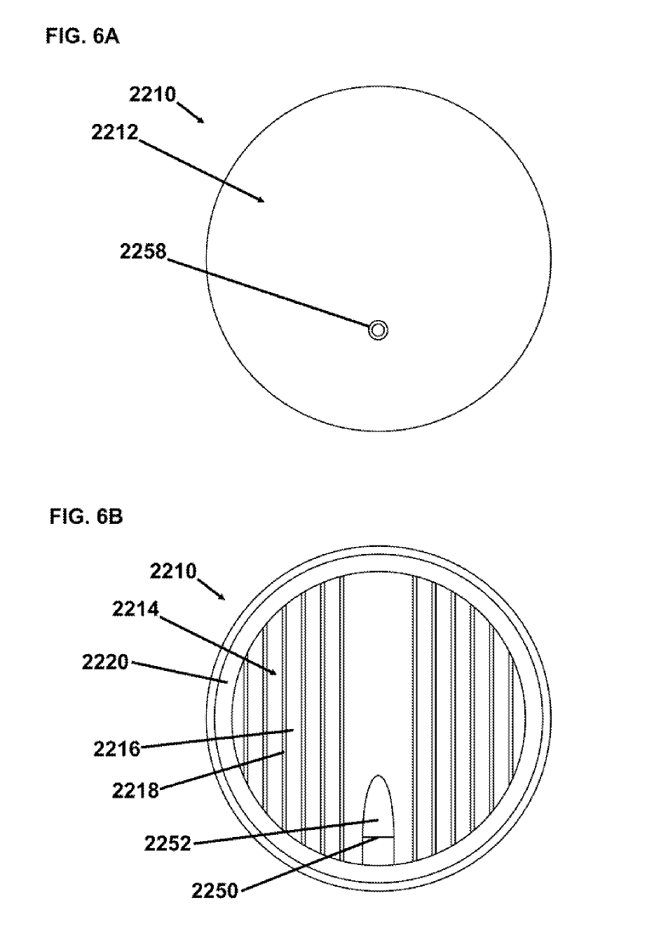

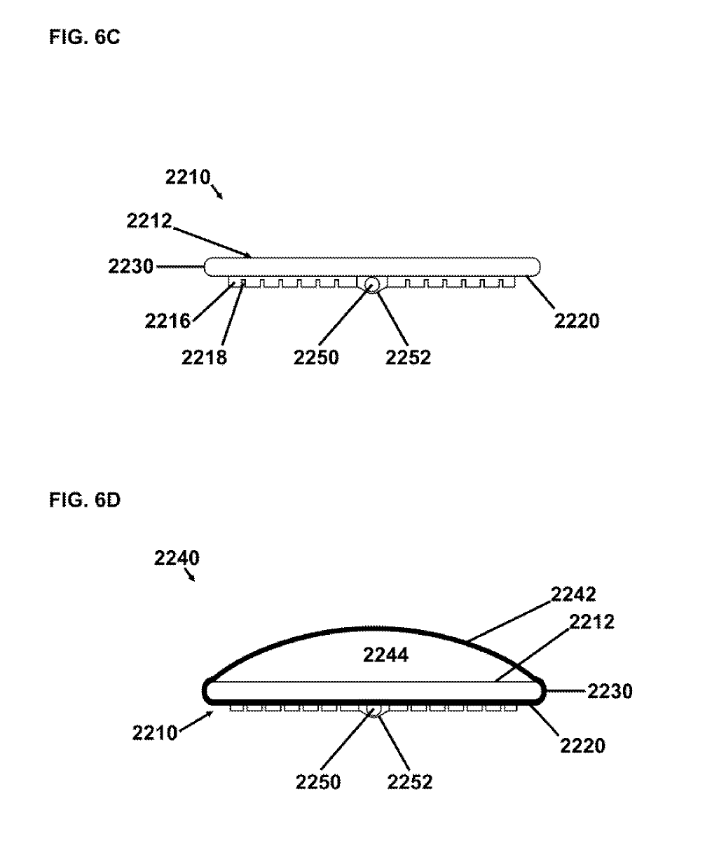

Aspects of the present specification disclose, in part, backing plate having a valve. The backing plate of the present specification may, or may not, comprise a valve. As used herein, the term “valve” is synonymous with “port”, “fill port”, or “fill valve”, and refer to a sealable orifice used as an access opening to introduce, contain, or remove a material, such as, e.g., a gas, a liquid, a gel, or a solid, into or from the interior chamber of the shell. The valve can be located on a side surface of the backing plate or on the back surface of the backing plate. A valve can be a check valve or one-way valve and includes, without limitation, a duckbill valve, a diaphragm valve, a reed valve, a leaf valve, a cross slit valve, or a plug valve. The valve can be positioned in a manner that locates the valve substantially flush with the surface of the backing plate (FIG. 5), or the valve can be housed in a protuberance (FIG. 6).

FIG. 5 illustrates a backing plate disclosed in the present specification including a valve flush with the back surface. FIG. 5A is a front view showing the front surface 2112 and conduit opening 2158 of the backing plate 2110. In some embodiments, an implant shell is affixed to all or a portion of front surface 2112. FIG. 5B is a back view showing the back surface 2114 of backing plate 2110 including the parallel ridges 2116 and grooves 2118, the outside rim 2120, and a valve 2150 that is flush with back surface 2114. FIG. 5C is an elevation view of the backing plate 2110 with ridges 2116 and grooves 2118. This view also shows side 2130 of backing plate 2110. In some embodiments, an implant shell is affixed to side 2130 of backing plate 2110 and/or outside rim 2120 of back surface 2114. FIG. 5D is an elevation view of a soft tissue implant 2140 comprising a shell 2142 having an interior chamber 2144 that is affixed to side 2130 of backing plate 2110 and/or outside rim 2120 of back surface 2114.

FIG. 6 illustrates a backing plate disclosed in the present specification including a valve housed in a protuberance. FIG. 6A is a front view showing the front surface 2212 and conduit opening 2258 of the backing plate 2210. In some embodiments, an implant shell is affixed to all or a portion of front surface 2212. FIG. 6B is a back view showing the back surface 2214 of backing plate 2210 including the parallel ridges 2216 and grooves 2218, the outside rim 2220, and valve 2250 housed in protuberance 2252. FIG. 6C is an elevation view of the backing plate 2210 and shows ridges 2216 and grooves 2218 as well as valve 2250 housed in protuberance 2252. This view also shows side 2230 of backing plate 2210. In some embodiments, an implant shell is affixed to side 2230 of backing plate 2210 and/or outside rim 2220 of back surface 2214. FIG. 6D is an elevation view of a soft tissue implant 2240 comprising a shell 2142 having an interior chamber 2244 that is affixed to side 2230 of backing plate 2210 and/or outside rim 2220 of back surface 2214.

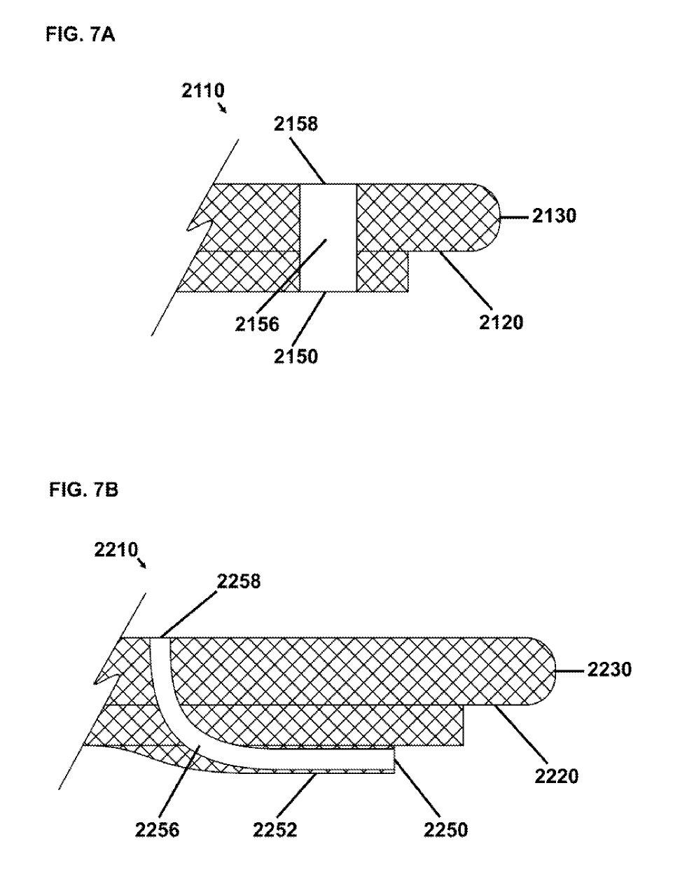

A conduit connects the valve to the front surface of the backing plate. A conduit is a channel ending in an opening (conduit opening) that enables the introduction or removal of a material, such as, e.g., a gas, a liquid, a gel, or a solid, into or from the interior chamber of an implant shell via an opening in the shell (FIG. 7). Although a conduit opening may be positioned anywhere on the front surface of a backing plate, it is typically located in proximity to the location of the value. FIG. 7 illustrates a conduit connecting a valve to the front surface of a backing plate. FIG. 7A is a cross-section view of the backing plate 2110 shows valve 2150 that is flush with back surface 2114 and conduit 2156 and the opening of the conduit opening 2158 on the front surface of the backing plate. FIG. 7B is a cross-section view of the backing plate 2210 shows valve 2250 housed in protuberance 2252 and conduit 2256 and the opening of the conduit opening 2258 on the front surface of the backing plate. The conduit may be of any size with the proviso that the size is sufficient to allow the introduction or removal of a material, such as, e.g., a gas, a liquid, a gel, or a solid, into or from the interior chamber of an implant shell via an opening in the shell. In aspects of this embodiment, the conduit size is at least 1 mm in diameter, at least 2 mm in diameter, at least 3 mm in diameter, at least 4 mm in diameter, at least 5 mm in diameter, at least 6 mm in diameter, at least 7 mm in diameter, at least 8 mm in diameter, at least 9 mm in diameter, or at least 10 mm in diameter. In other aspects of this embodiment, the conduit size is about 1 mm in diameter to about 10 m in diameter, about 2 mm in diameter to about 9 m in diameter, about 3 mm in diameter to about 8 m in diameter, or about 4 mm in diameter to about 7 m in diameter.

When incorporated with an implant, an opening in the shell of the implant is aligned with the conduit opening of the backing plate to allow introduction or removal of a material to the interior chamber of the shell. Additionally, the valve can be external to the backing plate, where the valve is connected to the backing plate via an external conduit like a flexible tube.

The backing plate disclosed in the present specification can be affixed to any soft tissue implant including prosthetic implants and tissue expanders. Affixing a backing plate to an implant can be accomplished by any suitable method, including, without limitation, gluing, bonding, melting, and/or any combination thereof. As a non-limiting example, a dispersion of silicone can be applied as an adhesive onto the front surface of a backing plate or a back surface of an implant, or both, and then the two materials placed together in a manner that allows the adhesive to attached the implant to the front surface of the backing plate. The silicone adhesive is then allowed to cure. Exemplary prosthetic implants that can be affixed to a backing plate disclosed in the present specification include, without limitation, prosthetic implants for a breast, pectoral area, upper arm (triceps and biceps), buttock, upper leg (quadriceps and hamstring), and calf.

Exemplary soft tissue implants that can be modified to incorporate the presently disclosed backing plate, mutatis mutandis, are described in, e.g., Andrews, et al., Implantable Prosthesis with Open Cell Textured Surface and Method for Forming Same, U.S. Pat. No. 5,545,220; Atala, Systems and Methods for Promoting Tissue Growth, U.S. Pat. No. 6,432,081, U.S. Pat. No. 6,287,275, U.S. Pat. No. 6,048,330, U.S. Pat. No. 5,858,003; Audretsch, Tissue Expanders, U.S. Pat. No. 4,950,292; Austad, Self-Inflating Tissue Expander, U.S. Pat. No. 5,005,591; Austad, Tissue Expander, U.S. Pat. No. 4,984,585; Bark and Falk, Tissue Expander, U.S. Pat. No. 5,141,508; Bark, et al., Tissue Expander and Method, U.S. Pat. No. 5,074,878; Bark, et al., Self-Sealing Tissue Expander and Method, U.S. Pat. No. 5,066,303; Bartholdson, Implantable Breast Prostheses, U.S. Pat. No. 4,507,810; Bartlett, et al., Soft Tissue Implants, U.S. Pat. No. 5,713,959; Baeke, Breast Implant with Position Lock, U.S. Pat. No. 6,203,570; Bellin, et al., Non-Rotating Breast Implant, U.S. Pat. No. 6,692,527, U.S. Pat. No. 7,105,116; Becker, Permanent Reconstruction Implant and Method of Performing Human Tissue Expansion, U.S. Pat. No. 4,643,733; Boyd, Implantable Prosthetic Device and Tethered Inflation Valve for Volume, U.S. Pat. No. 5,146,933; Brauman, Implantable Prosthetic Devices, RE35,391, U.S. Pat. No. 4,963,150, U.S. Pat. No. 4,820,303, U.S. Pat. No. 4,648,880; Brennan, System and Method for Breast Augmentation, U.S. Pat. No. 7,169,180; Bretz, Implant Device, U.S. Pat. No. 6,932,840; Brodniewicz, Biocompatible Surgical Implant, U.S. Pat. No. 5,630,842; Burnett, Method and Apparatus for Minimally Invasive Implants, U.S. 2009/0254179, U.S. 2007/0185575; Cao, Mammary Prosthesis Made of Polyacrylamide Hydrogel, U.S. Pat. No. 6,955,690; Cavon, Non-Enveloped Gel Prosthesis, U.S. Pat. No. 4,840,628; Cheng, et al., Thermally Reversible Implant, U.S. Pat. No. 7,160,931, U.S. 2007/0110784, U.S. 2004/0029994; Christensen and Ainpour, Prosthesis with Improved Biocompatibility, U.S. Pat. No. 5,116,371; Connell, Differential Tissue Expander Implant, U.S. 2007/0233273; Cox, Jr., Inflatable Implant, U.S. Pat. No. 4,969,899; Dreschnack and Villa, Breast Implant with Locatable Injection Site, U.S. 2003/0144734; Dubrul and Maxwell, Textured Tissue Expander, U.S. Pat. No. 5,092,348; Eaton, Flexible seamless memory tissue expanding implant, U.S. Pat. No. 6,315,796; Ersek, et al., Textured Silicone Implant Prosthesis, U.S. Pat. No. 4,955,909; Falcon, et al., Reinforced Radius Mammary Prostheses and Soft Tissue Expanders, U.S. Pat. No. 6,605,116; Fallot and Ftaiha, Breast Prosthesis, U.S. Pat. No. 6,156,066; Fields, Apparatus for Tissue Expansion Using Pulsatile Motion, U.S. Pat. No. 6,733,537; Frisch, Shape Retention Tissue Expander and Method of Using, U.S. Pat. No. 4,615,704; Gauger, Tissue Expander and Method of Making and Using, U.S. Pat. No. 4,899,764; Gedebou, Tissue Expander, System and Method, U.S. Pat. No. 7,238,193, U.S. Pat. No. 7,229,459, U.S. Pat. No. 7,226,463, U.S. Pat. No. 6,896,689; Georgiade, Mammary Prosthesis Fill and Method of Making Same, U.S. Pat. No. 5,219,360; Greenburg, et al., Method for Tissue Expansion Using Pulsatile Motion, U.S. Pat. No. 6,668,836; Guest, Tissue Expander with Protection Against Accidental Puncture, U.S. Pat. No. 6,743,254; Hamas, Mammary Prosthesis with Multiple Flow Spaces, U.S. Pat. No. 4,531,244; Henley, Fluid Filled Prosthesis Excluding Gas-Filled Beads, U.S. Pat. No. 5,534,023; Heyler, III, Spring Ring Tissue Expander, U.S. Pat. No. 4,828,560; Iverson and Trinter, Enhanced Surface Implant and Method of Manufacture, U.S. Pat. No. 5,964,803; Iverson and Trinter, Internally Configured Prosthesis, U.S. Pat. No. 5,961,552; Iverson, Laminated Prosthesis and Method of Manufacture, U.S. Pat. No. 5,935,164; Jakubczak, Multiple Envelope Tissue Expander Device, U.S. Pat. No. 4,651,717; Janzen, et al., Soft Tissue Implant, U.S. Pat. No. 5,922,024, U.S. Pat. No. 5,676,698; Jefferies and Spence, Breast Prosthesis with Improved Biocompatibility and Method of Making the Same, U.S. Pat. No. 4,772,284; Johnson, Solid Filled Implants, U.S. Pat. No. 6,544,287; Kesten and Lesh, Breast Implant with Adjustable Compression Response, U.S. 2009/0198328; Kesten and Lesh, Breast Implant With Internal Flow Dampening, U.S. 2009/0198329; Khouri, External Tissue Distraction with Expanding Frames, U.S. Pat. No. 6,699,176; Khouri, Method and Apparatus For External Tissue Distraction with Frame Having Membrane Applied with Surface Tension, U.S. Pat. No. 6,641,527; Khouri, Method and Apparatus for Expanding Soft Tissue with Shape Memory Alloys, U.S. Pat. No. 6,478,656; Khouri, Mechanical Soft Tissue Enlarger, U.S. Pat. No. 5,662,583; Knapp, et al., Hydraulic Foam Tissue Implant, U.S. Pat. No. 5,824,081; Kronowitz, Methods and Devices for Breast Reconstruction, U.S. 2008/0288068; Rosenberg, Double Chamber Tissue Expander, U.S. Pat. No. 5,630,843; LaForest and Taylor, Mammary Prosthesis Having Adjustable Projection, U.S. Pat. No. 4,605,412; Ledergerber, Tissue Expander, U.S. Pat. No. 6,228,116; Ledergerber, Implantable Prosthetic Device, U.S. Pat. No. 6,187,043, U.S. Pat. No. 5,383,929, U.S. Pat. No. 5,282,856, U.S. Pat. No. 4,955,907; Lesh, Tissue Augmentation Device, U.S. Pat. No. 7,641,688; Lesh, Systems and Devices for Soft Tissue Augmentation, U.S. Pat. No. 7,244,270; Lynn and Foglietti, Multiple Compartment Breast Prosthesis, U.S. Pat. No. 5,147,398, U.S. Pat. No. 5,092,882; Manders, et al., Dimensionally Adjustable Soft Tissue Expander and Method, U.S. Pat. No. 5,571,179; Maxwell, et al., Variable Cohesive Gel Form-Stable Breast Implant, U.S. 2007/0135916; Miller, Stacked Breast Implant, U.S. Pat. No. 5,236,454; Miller, Breast Implant, U.S. 2002/0038147; Muller, Mammary Prosthesis, U.S. Pat. No. 5,447,535; Nakao, Device for Providing a Portion of an Organism with a Desired Shape, U.S. Pat. No. 6,755,861; Oddsen and Ger, External Tissue Expansion Device for Breast Reconstruction, Male Pattern Baldness and Removal of Nevi and Keloids, U.S. Pat. No. 6,254,624; Perry and Maxwell, Medical Protheses Containing a Gel-Filler Comprising Principally Water and Cellulose Derivative, U.S. Pat. No. 5,531,786; Perry and Maxwell, Gel-Filled Implants, U.S. Pat. No. 5,282,857; Peterson, Encapsulated Implant, U.S. Pat. No. 5,246,454; Picha, Mammary Implant and Method for Reducing Capsule Contracture, U.S. Pat. No. 5,236,453; Picha, Implant with Textured Surface, U.S. Pat. No. 5,207,709; Picha, Tissue Expander and Method for Expanding Tissue, U.S. Pat. No. 5,158,571; Pierce and Wisniewski, Inflatable Surgical Implant with Variable Inflation Position, U.S. Pat. No. 4,662,357; Perouse, Reconstruction Implant, U.S. Pat. No. 6,387,133; Perouse, Breast Prosthesis, U.S. Pat. No. 5,843,189; Purkait, Breast Implants and Methods of Manufacture, U.S. Pat. No. 7,625,405, U.S. 2010/0137985; Quaid, Mammary Implant Having Shell with Unitary Rough-Textured Outer Layer, U.S. Pat. No. 5,674,285; Quaid, Open-Cell, Silicone-Elastomer Medical Implant, U.S. Pat. No. 5,007,929; Raphael and Harris, Lip Implant, Instrumentation and Method for Insertion, U.S. Pat. No. 7,329,286; Rosenberg, Balloon-Type Tissue Expansion Device, U.S. Pat. No. 4,800,901; Rosenberg, Tissue Expander Apparatus, and Methods of Constructing and Utilizing Same, U.S. Pat. No. 5,425,760; Sakura, Jr., Prosthetic Tissue Implant and Filler Therefor, U.S. Pat. No. 6,099,565; Sasaki, et al., Tissue Expander and Method of Making and Using, U.S. Pat. No. 4,841,992; Schuessler, Self-Sealing Shell for Inflatable Prostheses, U.S. 2010/0049317; Schuessler, Self-Sealing Shell for Inflatable Prostheses, U.S. 2010/0049316; Shalon, et al., Tissue Expansion Devices, U.S. 2010/0010531, U.S. 2006/0069403; Snyder, Jr., Multiple Section Breast Prosthesis, U.S. Pat. No. 5,902,335; Stephens and Martin, Inflatable Implant, U.S. Pat. No. 7,632,291; Tiffany and Winn, Rupture-Resistant Prosthesis with Creasable Shell and Method of Forming Same, U.S. Pat. No. 4,731,081; VanBeek and Iverson, Mechanical Tissue Expander, U.S. Pat. No. 5,882,353; Van Dyke, et al., Implantable Prosthetic or Tissue Expanding Device, U.S. Pat. No. 6,849,092, U.S. Pat. No. 6,371,984; Watson, et al., Tissue Expanding Device and Method of Making Same, U.S. Pat. No. 4,823,815; Waybright and Carlisle, Adjunctive Filler Material for Fluid-Filled Prosthesis, U.S. Pat. No. 5,549,671; Wiese, Tissue Expander Inflating Due to Osmotic Driving Forces of a Shaped Body of Hydrogel and an Aqueous Solution, U.S. Pat. No. 5,496,368; Yan and Purkait, Textured Surface Prosthesis Implants, U.S. Pat. No. 4,960,425; Young, Combination Dissector and Expander, U.S. Pat. No. 5,984,943, U.S. Pat. No. 5,871,497, U.S. Pat. No. 5,855,588, U.S. Pat. No. 5,776,159; and Zucherman, et al., Implantable Prosthetic or Tissue Expanding Device, U.S. Pat. No. 6,783,546; each of which is incorporated by reference in its entirety for the purpose of describing and disclosing soft tissue implant.

Aspects of the present specification disclose, in part, a soft tissue implant comprising a shell including an interior chamber. The shell can be a self-sealing shell in which case a valve is not required, or the shell can not be a self-sealing shell in which case a valve as disclosed in the present specification may be a necessary component. Like the backing plates, a shell disclosed in the present specification can be composed of any substantially non-degradable and biocompatible material, including, without limitation, the elastomers disclosed in the present specification. Additionally, the shell can have an untextured surface or a textured surface. A textured surface is formed by imprinting a pattern onto the surface, or the textured surface can be made from a porous material. Exemplary examples of porous material are described in, e.g., Goraltchouk, et al., Implantable Materials and Methods for Producing Same, U.S. Provisional Patent Application 61/301,104, filed Feb. 3, 2010; Liu, et al., Porous Materials, Methods of Making and Uses, U.S. Provisional Patent Application 61/333,120, filed May 10, 2010; Manesis, et al., Silicone Implant with Imprinted Texture, U.S. Provisional Patent Application 61/333,146, filed May 10, 2010; Liu, et al., Porous Materials, Methods of Making and Uses, U.S. Provisional Patent Application 61/333,613, filed May 11, 2010; and Thompson, et al., Open-Cell Surface Foam Materials, U.S. Provisional Patent Application 61/333,146, filed Jun. 16, 2010; each of which is incorporated by reference in its entirety for the purpose of describing and disclosing porous materials.

Thus, in one embodiment, an implant comprises a self-sealing shell including an interior chamber and a backing plate disclosed in the present specification that lacks a valve. In another embodiment, an implant comprises a shell including an interior chamber and a valve and a backing plate disclosed in the present specification that lacks a valve. In yet another embodiment, an implant comprises a shell including an interior chamber and a backing plate disclosed in the present specification including a valve, where a conduit connects the backing plate valve and the interior chamber of the shell to allow introduction, containment, or removal of a material, such as, e.g., a gas, a liquid, a gel, or a solid, into or from the interior chamber of the shell.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to a backing plate comprising a front surface and a back surface comprising a plurality of parallel ridges and grooves, and soft tissue implants comprising such backing plates.

### Example 1

### Backing Plate

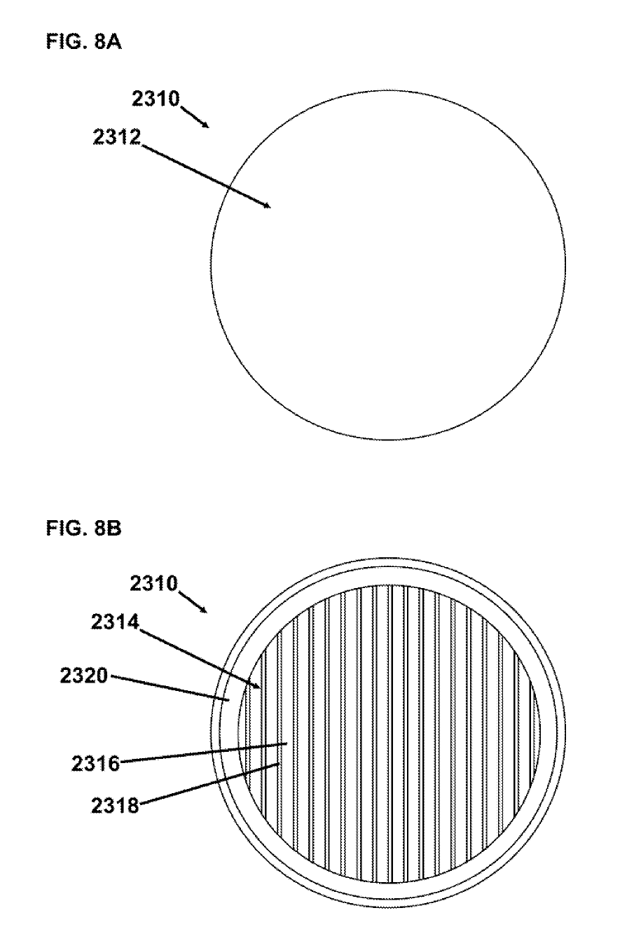

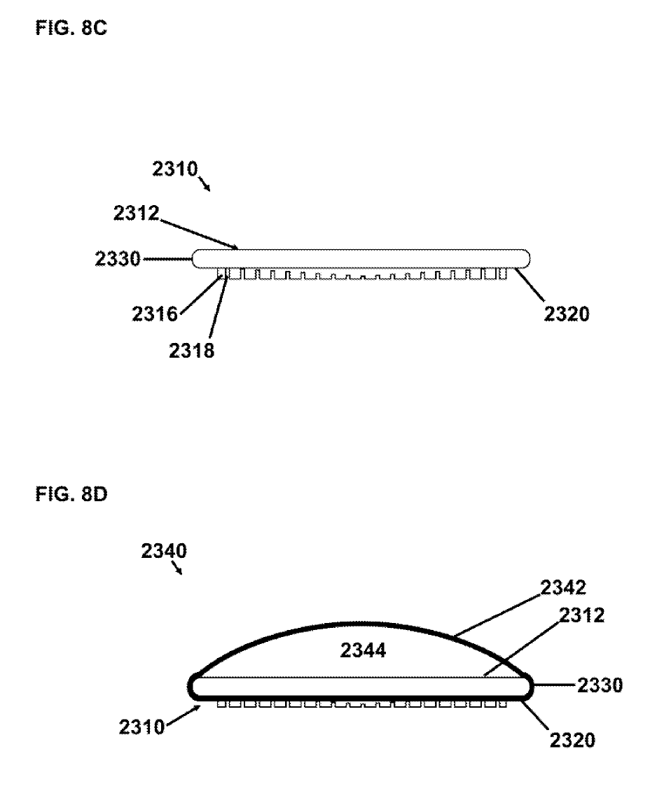

This example illustrates a backing plate as disclosed in the present specification depicting a plate without a valve (FIG. 8). FIG. 8A is a front view showing the front surface 2312 of the backing plate 2310. FIG. 8B is a back view showing the back surface 2314 of backing plate 2310 including the parallel ridges 2316 and grooves 2318 and the outside rim 2320. FIG. 8C is an elevation view of the backing plate 2310 with ridges 2316 and grooves 2318. This view also shows side 2330 of backing plate 2310. FIG. 8D is an elevation view showing a soft tissue implant 2340 comprising a shell 2342 having an interior chamber 2344 that is affixed to side 2330 of backing plate 2310 and/or outside rim 2320 of back surface 2314. In other embodiments, the front surface 2312 serves as an attachment surface used to affix implant shell 2342.

### Example 2

### Backing Plate

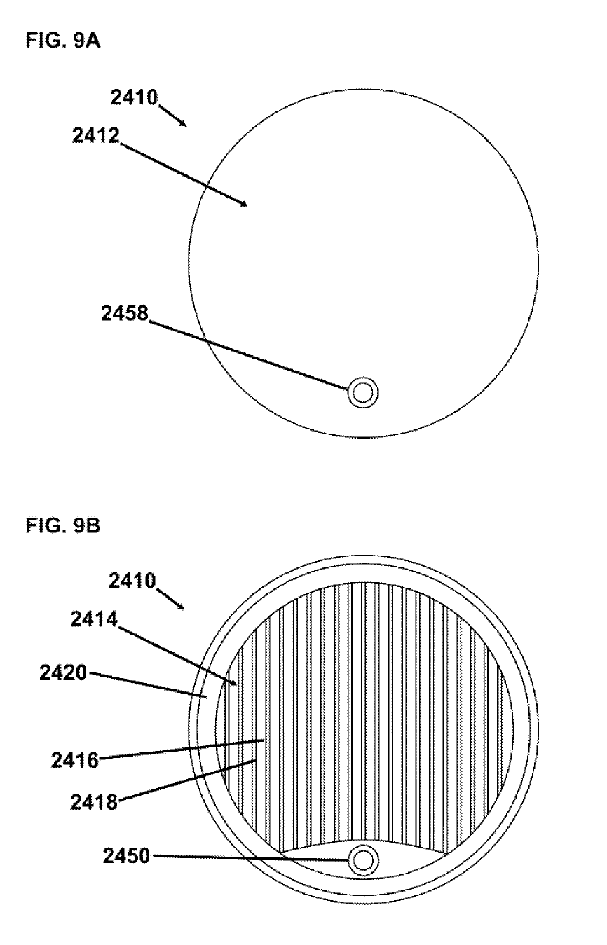

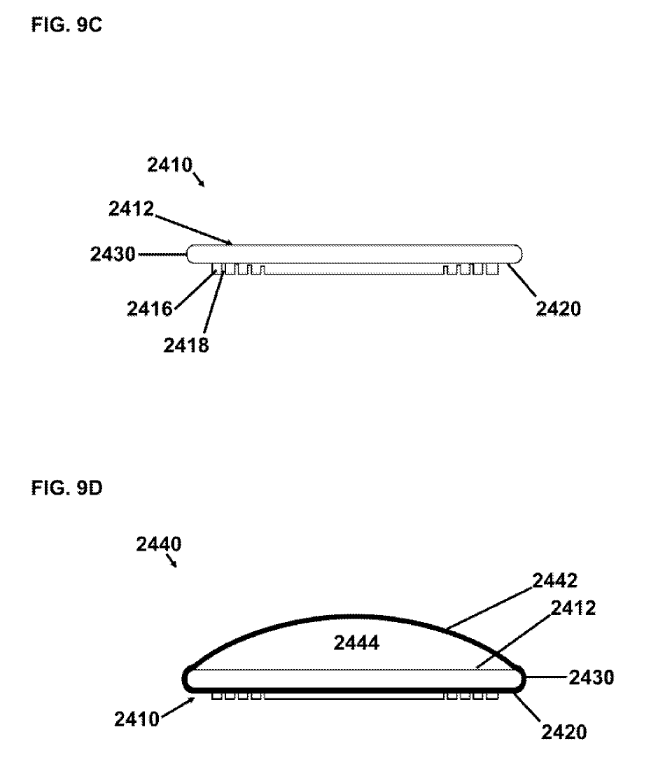

This example illustrates a backing plate as disclosed in the present specification depicting a plate with valve substantially flush with the back surface (FIG. 9). FIG. 9A is a front view showing the front surface 2412 and conduit opening 2458 of the backing plate 2410. FIG. 9B is a back view showing the back surface 2414 of backing plate 2410 including the parallel ridges 2416 and grooves 2418, the outside rim 2420, and a valve 2450 that is substantially flush with back surface 2414. FIG. 9C is an elevation view of the backing plate 2410 with ridges 2416 and grooves 2118. This view also shows side 2430 of backing plate 2410. FIG. 9D is an elevation view of a soft tissue implant 2440 comprising a shell 2442 having an interior chamber 2444 that is affixed to side 2430 of backing plate 2410 and/or outside rim 2420 of back surface 2414. In other embodiments, the front surface 2412 serves as an attachment surface used to affix implant shell 2442.

### Example 3

### Backing Plate

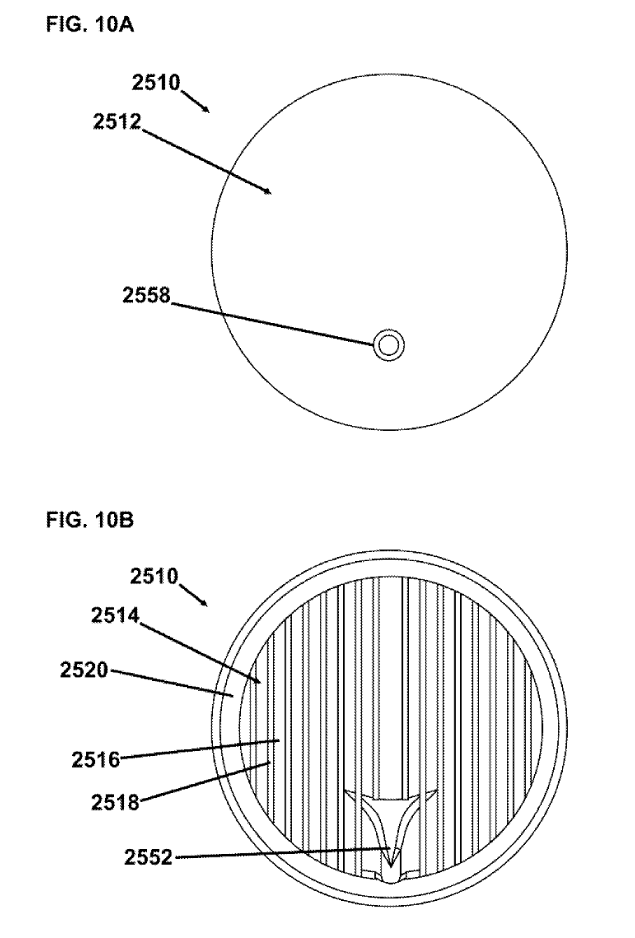

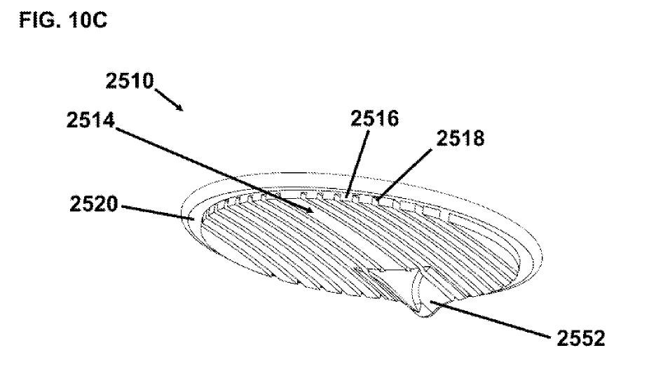

This example illustrates a backing plate as disclosed in the present specification depicting a plate with valve housed in pertuberance (FIG. 10). FIG. 10A is a front view showing the front surface 2512 and conduit opening 2558 of the backing plate 2510. FIG. 6B is a back view showing the back surface 2514 of backing plate 2510 including the parallel ridges 2516 and grooves 2518, outside rim 2520, and valve 2550 housed in protuberance 2552. FIG. 10C is a perspective view of the backing plate 2510 and shows back surface 2514 with ridges 2516 and grooves 2518 as well as outside rim 2520 and protuberance 2552.

### Example 4

### Backing Plate

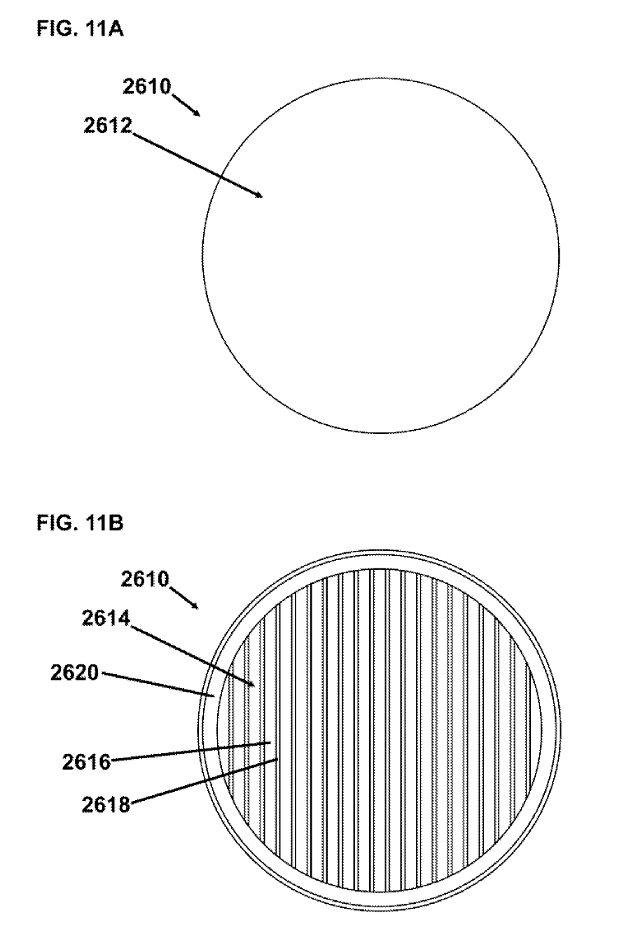

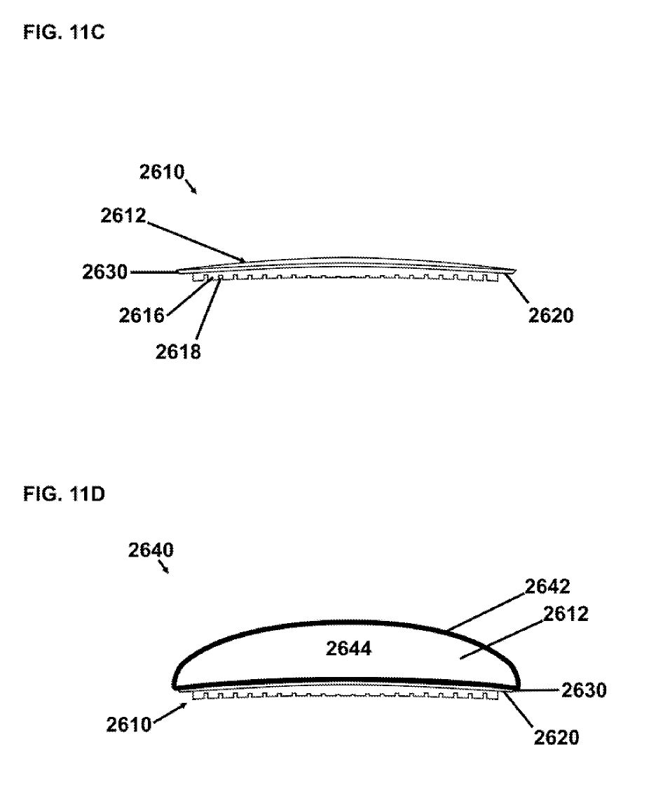

This example illustrates a backing plate as disclosed in the present specification depicting a plate without a valve and having a concave planar shape (FIG. 11). FIG. 11A is a front view showing the front surface 2612 of the backing plate 2610. FIG. 11B is a back view showing the back surface 2614 of backing plate 2610 including the parallel ridges 2616 and grooves 2618 and the outside rim 2620. FIG. 11C is an elevation view of the backing plate 2610 with ridges 2616 and grooves 2618. This view also shows side 2630 of backing plate 2610. FIG. 11D is an elevation view showing a soft tissue implant 2640 comprising a shell 2642 having an interior chamber 2644 that is affixed to front surface 2612 of backing plate 2610. In other embodiments, side 2630 of backing plate 2610 and/or outside rim 2620 of back surface 2614 serves as an attachment surface used to affix implant shell 2642.

### Example 5

### Backing Plate

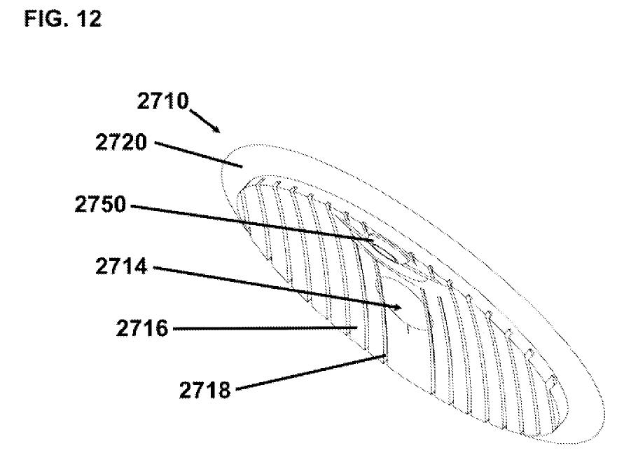

This example illustrates a backing plate as disclosed in the present specification depicting a plate with valve substantially flush with the back surface and having a concave planar shape (FIG. 12). FIG. 12 is a perspective view of the backing plate 2710 and shows back surface 2714 with ridges 2716 and grooves 2718 as well as outside rim 2720 and valve 2750.

In closing, it is to be understood that although aspects of the present specification have been described with reference to the various embodiments, one skilled in the art will readily appreciate that the specific examples disclosed are only illustrative of the principles of the subject matter disclosed in the present specification. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A breast prosthesis comprising:
a elastic anterior portion; and
a backing plate, secured to the anterior portion and forming a posterior portion of the prosthesis, the backing plate comprising a surface defined by a plurality of spaced apart ridges.

2. The prosthesis of claim 1, wherein the spacing between the ridges is varied.

3. The prosthesis of claim 2, wherein the varied spacing between the ridges is irregular.

4. The prosthesis of claim 1, wherein the ridges are of different heights.

5. The prosthesis of claim 1, wherein the height of each of the ridges is at least 0.5 mm.

6. The prosthesis of claim 1, wherein the height of each of the ridges is from about 0.5 mm to about 5 mm.

7. The prosthesis of claim 1, wherein the ridges are of different widths.

8. The prosthesis of claim 1, wherein the width of each of the ridges is at least 0.5 mm.

9. The prosthesis of claim 1, wherein the width of each of the ridges is from about 0.5 mm to about 5 mm.

10. The prosthesis of claim 1, wherein the backing plate has a flexibility between the ridges to enable the backing plate to form a substantially rolled configuration.

11. The prosthesis of claim 1, wherein the backing plate further comprises a valve.

12. The prosthesis of claim 1 in the form of a tissue expander.

13. A breast prosthesis comprising:
a fillable, elastic anterior portion; and
a flexible backing plate, secured to the anterior portion and forming a posterior portion of the prosthesis, the anterior portion and the backing plate defining a fluid-fillable cavity therebetween;
the backing plate comprising a surface defined by a plurality of alternating ridges, the ridges having at least one of varied heights or varied widths and having a flexibility between the ridges to enable the backing plate to form a substantially rolled configuration;
the backing further comprising a valve for facilitating filling of the chamber.
